(19) United States
(12) Patent Application Publication
(10) Pub. No.: US 2021/0015500 A1
(43) Pub. Date: Jan. 21, 2021

(54) SYSTEM AND METHOD FOR PLACING FASTENERS INTO INTRAMEDULLARY NAILS

(71) Applicant: ALEXANDER NASSONOV, Kiryat Shmona (IL)

(72) Inventor: ALEXANDER NASSONOV, Kiryat Shmona (IL)

(21) Appl. No.: 16/514,014

(22) Filed: Jul. 17, 2019

(51) Int. Cl. A61B 17/17; A61B 17/72

(52) U.S. Cl. CPC A61B 17/1725; A61B 2017/564; A61B 17/1703; A61B 17/7233

(57) ABSTRACT

A new system and a method for aligning an axis of radiation of fluoroscopic station, an axis of aiming guide and an axis of at least one hole in a distal portion of an intramedullary nail are proposed. The system consists of the fluoroscopic station, inclinometers and a targeting jig, having a positioning module adjustable relatively to the jig to reach the anticipated position, whereas the jig is attached to a proximal end of the IM nail and the aiming guide with radiopaque pattern attached to the positioning module, wherein the inclinometers are attached to a jig base and to the fluoroscopic station. The system and the method according to this invention provide and then maintain fast and accurate positioning of a drill bit guide in coincidence with at least one of IM nail holes.

### FIELD OF THE INVENTION

This invention relates to orthopaedic navigation system and method, for establishing a desired orientation of a navigated element. System and method make aligning of an axis of radiation of a fluoroscopic station and an axis of at least one hole in a distal portion of an intramedullary nail simple and correct.

### DESCRIPTION OF RELATED ARTS

In repairing broken bones, and particularly long bones of the body such as femur, tibia or humerus, a common surgical practice requests to insert a metal rod known as an intramedullary nail (IM nail) into the interior portion of the bone. The IM nail is then secured in place with respect to the bone by installing one or more locking screws or bolts at the proximal and distal portions of the IM nail.

In order to install the screws into distal blind holes of the IM nail and bone, a screw hole must be drilled transverse to the bone in direct alignment with each transverse hole in the IM nail. This drilling is performed after the IM nail has been inserted into the bone canal. It is desirable that drilling should pass cleanly through the transverse holes, to allow further correct insertion of the locking screws and to prevent metal shreds and shavings from being formed when the drill touches the nail. The nail's deformation due to stress during insertion into the bone intramedullary canal makes accurate positioning of the distal drilling and insertion of the locking screws very difficult.

Distal locking in most cases is performed with a fluoroscopic station (professionally called C-Arm) and traditionally uses “Free hand technique” that involves intensive use of X-Rays which pose well-known dangers from cumulative exposure. Both the surgeon and the patient are exposed to X-ray radiation from the fluoroscopy equipment used in the range of 400-1000 rem over the duration of the operation.

Different ancillary sighting devices (disclosed in U.S. Pat. Nos. 4,803,976; 6,129,729; 6,635,061; 6,656,189) do not actually solve the problem. Time of surgery strictly depends on skill of a surgeon and sometimes ended without positive result (without insertion of the distal locking screws and final desirable stabilization of the fractured bone).

Several methods of locating blind holes using all-mechanical devices were invented (U.S. Pat. Nos. 4,865,025; 6,027,506; 6,039,742). To adjust the position of drill guide after the nail distortion caused by bending, they use additional correction rod and drilling through the bone. These devices still have not shown sufficient results.

Optic-mechanical device (disclosed in U.S. Pat. No. 5,540,691) is based on a well-known partial transparency of human body to light. The device has no means to locate and affix correct position of drill bit and actually proposes a method similar to “Free hand technique” but using different sources of visible and non-visible radiation. The means to get precise position of drill bit are not described by the authors.

The problem of distal locking still exists despite that known systems and devices can locate said blind holes and assist to place fasteners. Said devices such as ModAd and SureLock of De Puy Synthes Products Inc., two distal targeting devices of Stryker Trauma GmbH and Trigen Sureshot system of Smith&Nephew provide solution for IM nails but produced only by said companies and clinical results depend a lot to qualification of the operational room personnel.

### SUMMARY OF THE PROPOSED INVENTION

A new system and a method for aligning an axis of radiation of a fluoroscopic station, an axis of aiming guide and an axis of at least one hole in a distal portion of an intramedullary nail are proposed. The system consists of the fluoroscopic station, inclinometers and indicator-spike inserted in a segment of a broken bone where said holes should be drilled. The system further consists of a targeting jig having a positioning module adjustable relatively to the jig, whereas the jig is attached to the proximal end of the IM nail by an insertion handle, and an aiming guide with radiopaque structure attached to the positioning module wherein the inclinometers are attached to the jig base and to the fluoroscopic station. The system and the method provide fast and accurate positioning of the drill bit guide cannula in coincidence with at least one of IM nail holes.

The targeting jig can be preliminary configured in compliance with any existing IM nail of any producer using four threaded knobs sliding along dimensional scales. The jig's correct configuration, according to the chosen nail, can be stored as a four numbers sequence indicating necessary positions of said four knobs relatively said scales. Then every IM nail will have said unique set of numbers and to accommodate the jig to chosen nail there will be need just to move said knobs into position marked by said numbers. The jig is designed in such a configuration that, after all knobs are set and locked in desired positions, it converts from flexible to completely rigid structure. This feature helps surgery room personnel easily adjusting of the jig according to the chosen IM nail.

To estimate deviation of the distal holes of IM nail from the initial position using the fluoroscopic station, after insertion into medullary cavity, the aiming guide must be placed in line with central axis of radiation beam.

After the hole of aiming guide has been placed in correct position, drilling of the holes and inserting the locking screws goes according to traditional methods. During drilling and inserting of locking screw surgeon have to maintain the mutual position of the jig and the segment of broken bone by taking in account the position of indicator-spike.

### BRIEF DESCRIPTION OF THE DRAWINGS

In order to understand the subject matter and to see how it may be carried out in practice, embodiments will now be described with reference to the accompanying drawings, in which:

FIGS. 1 and 1A are schematic perspective views of the system ready to drill the distal holes inside the bone (the distal portions of the bone has partial cut to show the distal holes of the nail) and an enlarged portion of said view, respectively.

FIG. 2 is schematic perspective view of the targeting jig mounted on the intramedullary nail according to the present application;

FIG. 2 A is schematic perspective view of the insertion handle;

FIG. 3 is a side view of the arm of the targeting jig;

FIGS. 3 A and 3B are section and detailed views of said arm, respectively;

FIG. 4 is schematic perspective view of the positioning module with the aiming guide inserted in the guiding hole of the jig;

FIG. 4A and FIG. 4B is schematic perspective views of the xy-block and the y-rails block with the threaded y-knob and the aiming guide;

### DETAILED DESCRIPTION OF PREFERRED EMBODIMENTS

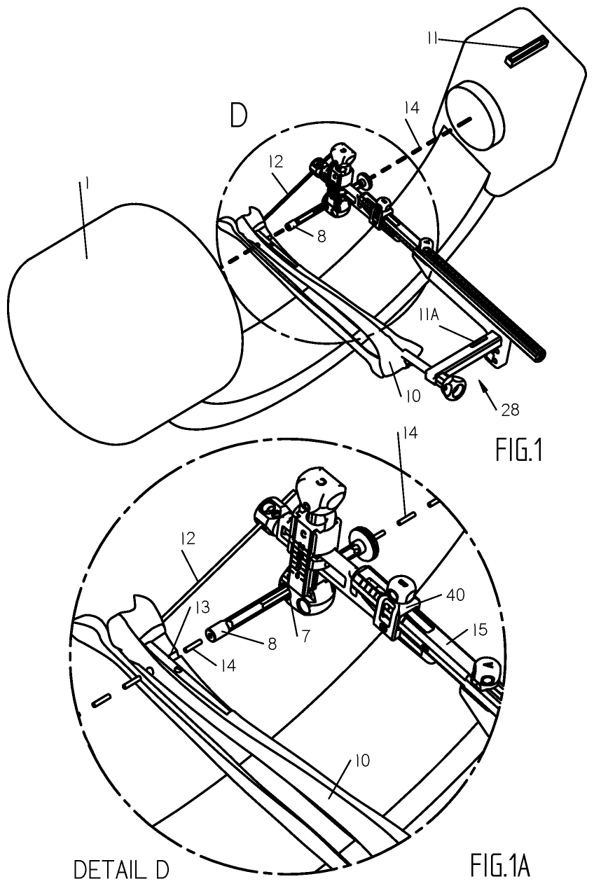

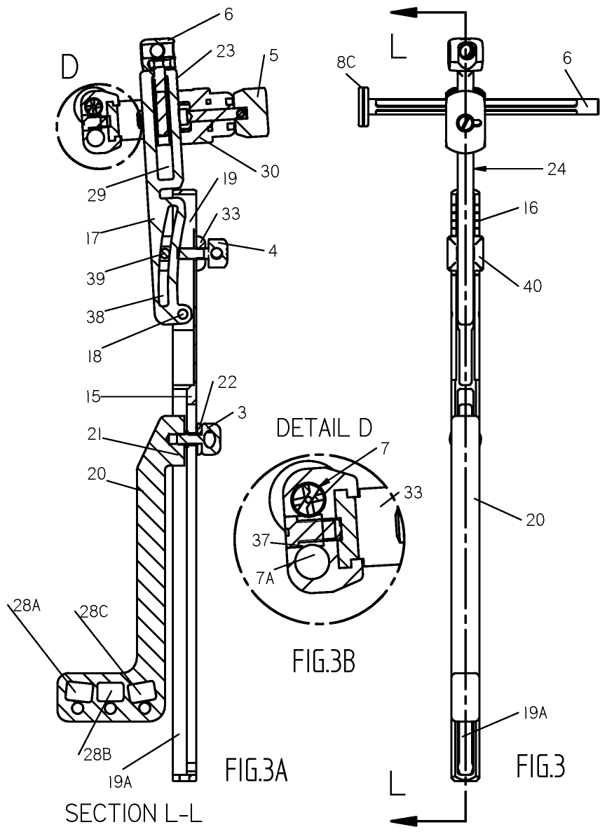

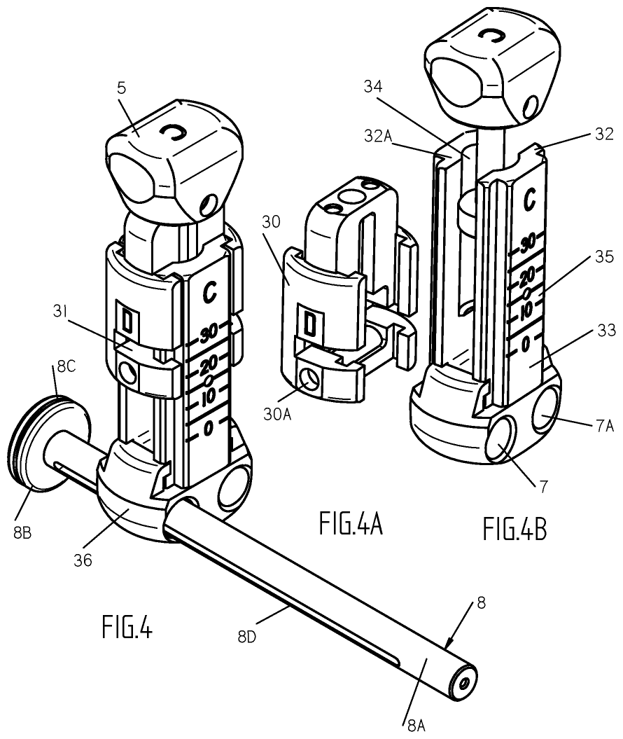

The present invention comprises the system (FIG. 1, FIG. 1A) for placing fasteners into distal holes of intramedullary nails, which has been implanted in a patient's bone, comprising the fluoroscopic station 1, the targeting jig (FIG. 3) according to this invention having threaded knobs 2, 3, 4, 5, 6 to adjust and maintain mutual position of parts, a guiding hole 7, 7A to adopt an aiming guide 8 and further the positioning module (FIG. 4) to change position of the guiding hole 7. The targeting jig (FIG. 3) further connected to the intramedullary nail 9 that implanted into the medullary cavity of the bone 10, whereas inclinometers 11, 11A placed on the fluoroscopic station 1 and the targeting jig (FIG. 3). The indicator-spike 12 attached to the portion of broken bone 10 where the holes will be drilled through a structure of the targeting jig.

The new method for locating distal holes 13 in an intramedullary nail 9 that has been implanted in a broken bone 10 of patient, using the targeting jig (FIG. 3) according to this invention, comprising following sequence of steps:
adjusting a shape of the targeting jig (FIG. 3) according to a set of stored numbers indicating positions of threaded knobs 2, 3, 4, 5, 6 to change configuration of the jig (FIG. 3) according to the shape of chosen intramedullary nail while maintaining an axis of a chosen distal hole and an axis of the guiding hole 7 parallel;
implanting the intramedullary nail 9 into the broken bone 10 and affixing to the bone by fasteners (not shown) through proximal holes (not shown) using regular methods;
providing visual control of the desired position between the broken bones 10 portions, the nail 9 and the jig (FIG. 3) by inserting through the jig structure with guaranteed gap the indicator-spike 12 into the portion of broken bone where distal holes will be drilled and further placing the targeting jig (FIG. 3) between the fluoroscopic station 1 and the broken bone 10 of patient;
using the inclinometers 11, 11A to place an axis of radiation beam 14 of radioscopy device 1 and an axis of one of distal holes 13 of intramedullary nail 9 in parallel;
inserting an aiming guide 8 with radiopaque marking 8C into the jig's guiding hole 7 and moving the fluoroscopic station 1 to place the axis of radiation beam 14 and the axis of said aiming guide 8 in line and then taking a picture of mutual position of the distal hole 13 and the aiming guide 8;
correcting according to the picture, by positioning module (FIG. 4), if necessary, the position of aiming guide 8 with regards to the targeting jig (FIG. 3), to place the axis of aiming guide 8 and axis of said distal hole 13 in line;
changing the aiming guide 8 to a drilling guide (not shown) and placing fasteners (not shown) into the bone through the distal holes 13 of the intramedullary nail 9 using regular methods.

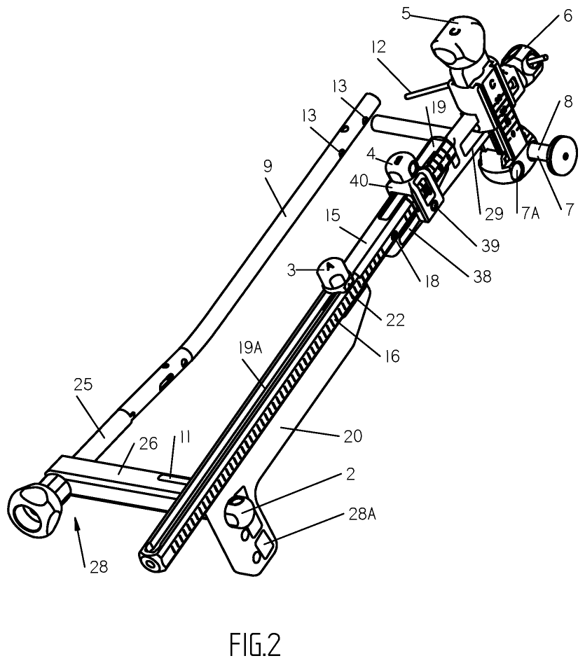

According to the invention, the new targeting jig (FIG. 2) consists of an insertion handle (FIG. 2A) affixed to a proximal end of the intramedullary nail 9, an arm (FIG. 3, FIG. 3A, FIG. 3B) comprising a rigid beam 15 with a measuring scale 16 connected to a hinge 17, attached to the beam by an axle 18 and placed into a slit 19 made inside of said beam 15 with possibility of rotation at said axle 18. The arm, further, has a beam-base 20 that has a lip 21 inserted into the slit 19A, where the lip 21 can slide inside this slit 19A along measuring scale 16. The beam 15 affixed between two dove tail joints where the first made in the beam base 20 and the second in a clump 22 that pinned down to the beam base 20 by threaded knob 3 protruding through said clump 22 and lip 24.

The arm is built in consideration that all moving parts of the arm move in parallel to the same plane and this plane is perpendicular to axis of aiming hole 7 of the intramedullary nail.

The insertion handle (FIG. 1A) consists further of a base stem 25 inserted in a lever 26 that connected to the beam base 20 by mortise-tenon joint wherein beam base 20 and the lever 26 placed perpendicularly to each other and affixed by the threaded knob 2. Said beam-base 20 has a set of mortise orifices 28 to adopt the jig FIG. 1 to different intramedullary nails. The jig could be attached to an insertion handle by any producer equipped with an adapter having the tenon to connect to beam-base 20 by said insertion handle, as well.

The inclinometers must be attached to the fluoroscopic station's emitter-receiver portion and to the jig. With help of inclinometers the axis of radiation beam of fluoroscopic station 14 and axis of chosen guiding hole 7 must be placed perpendicular to the hinge's plane 24. To achieve this task the aiming guide 8 (FIG. 4) comprised a rod 8A with a central hole to accommodate a metallic spike or a drill bit and the disk 8B with radiopaque ring 8C, placed at one of ends of said rod, whereas the rod and the disk made of radiolucent material and cuts 8D made along said rod, whereas the disk placed between the radiolucent device 1 and the jig (FIG. 3).

As mentioned before, the arm (FIG. 2) has the hinge 17 with a slot 29 in which inserted the xy-block 30 (FIG. 4A) having slots to accommodate both the x-rail 31 and y-rails 32, 32A where a y-rails block 33 (FIG. 4B) has y-rails on both sides of said block 30 and said hinge 17. The y-rail block 33 has a thread 34 to interact with the threaded y-knob 5 that drives the y-rails block 33 relatively to the xy-block 30. The xy-block 30 together with the y-rails block 33 driving along the slot 29 in said hinge 17 with help of a threaded hole 30A and the x-knob 6. The y-rails block 33 has a measuring scale 35 and guiding holes 7, 7A perpendicular to the side plane 24 of the hinge 17 with measuring scale 35A at the upper side, to accept tools such as the aiming guide 8 or a trocar.

A portion 36 of the y-rails block 33 having said guiding holes 7, 7A, could be made detachable to comply with different intramedullary nails having different sets of distal holes. To avoid accidental fall of tools placed inside guiding holes, a bore 37 with a diameter protruding inside said holes is made between said guiding holes 7, 7A and a spiral flat spring (not shown) inserted inside of said bore. The spiral spring (not shown) pushes the tools aside and so prevents of sliding said tools outside the holes.

The hinge 17 within the portion that can slide inside the slit 19 in the distal portion of the beam has an additional distal, bowed or slant, slot 38. A tow pin 39 inserted then through a slider 40 and said slot 38, and the slider 40 can shift along the beam 15 together with the tow pin 39 that moves inside said slot 38, initiating rotation of the hinge 17 on the axle 18. The slider 40 could be pinned opposite the hinge 17 by a threaded slider-knob 4 attached to said slider 40.

The new aiming jig (FIG. 1) provides ability to place guiding hole 7 in line with distal hole 13 of many different shaped intramedullary nails by using special procedure with help of the new targeting jig that described in detail above.

The present invention is defined by the appended claims:

1. A system for placing fasteners into distal holes of intramedullary nails, which has been implanted in a patient, comprising a fluoroscopic station, a targeting jig, according to this invention, having threaded knobs to adjust and maintain mutual position of parts, a positioning module with guiding hole to adopt an aiming or a drilling guide and the targeting jig further connected to an intramedullary nail that implanted into a medullary cavity of a broken bone, whereas inclinometers placed on the fluoroscopic station and the targeting jig and further an indicator-spike attached to the portion of broken bone where the holes will be drilled.

2. A method for locating distal holes in the intramedullary nail that has been implanted in the broken bone of patient, using the targeting jig according to this invention, comprising following sequence of steps:
(a) adjusting a shape of the targeting jig according to a set of stored numbers indicating positions of threaded knob to change configuration of the targeting jig according to a shape of chosen intramedullary nail while maintaining an axis of a chosen distal hole and an axis of the guiding hole parallel;
(b) implanting the intramedullary nail into the broken bone and affixing to the bone by fasteners through proximal holes using regular methods;
(c) providing visual control of a desired position between the broken bone's portions and the targeting jig, that affixed at a proximal end of the intramedullary nail, by imbedding the indicator-spike into the portion of broken bone, where fasteners will be inserted, through the jig structure with guaranteed gap, and further placing the targeting jig between emitter and receiver of the fluoroscopic station;
(d) using the inclinometers to place an axis of radiation beam of the fluoroscopic station and an axis of one of the distal holes of intramedullary nail parallel;
(e) inserting the aiming guide with radiopaque pattern into the targeting jig's guiding hole and moving the fluoroscopic station in position where the axis of radiation beam will be sited in line with the axis of said aiming guide and then taking a picture of mutual position of the distal hole and the aiming guide;
(f) correcting, if necessary, the position of aiming guide according to the picture, with regards to the targeting jig, by the positioning module, implemented into the said targeting jig, to place the axis of aiming guide and axis of said distal hole in line;
(g) changing the aiming guide to a drilling guide and placing fasteners into the bone through the distal holes of intramedullary nail using regular methods.

3. The targeting jig according to claim 1 for locating holes in an intramedullary nail, which has been implanted in a patient, comprising an arm, consisting of a rigid beam, with a slit and a measuring scale made along its length, a hinge, having an measuring ruler, attached to the beam inside said slit by an axle, a distal and a proximal slots, to accommodate the positioning module with guiding holes in the former and a tow pin in the latter, where the pin affixed further to a slider that can shift along the beam together with the tow pin that moves inside said proximal slot initiating rotation of the hinge on the axle, whereas the hinge and the slider could be affixed to the beam by a threaded slider-knob inserted into said slider;

4. The jig of claim 3 wherein the beam placed between two dove-tail joints, whereas the first one made in the beam-base attached to the beam with help of a lip inserted into the slit, where the lip slides inside this slit along the beam's measuring scale, and the beam-base affixed further to an intramedullary nail by an insertion handle, and the second one is made in a clump, that pinned down to the beam by the threaded clump-knob protruding through said clump and lip;

5. The jig of claim 4 wherein the insertion handle comprises a stem attached to a lever and affixed further to a proximal end of the intramedullary nail by a bolt, the lever further connected by the threaded lever-knob and a mortise-tenon joint to the beam base, whereas said beam base has a set of mortise orifices to adopt the jig to different intramedullary nails;

6. The jig of claim 5 where a jig comprising a regular insertion handle by any producer equipped with an adapter having the tenon to connect to beam-base by said insertion handle;

7. The jig of claim 5 wherein positioning module consists of a xy-block, a y-rails block having two sets of y-rails divided by a cut, so y-rails can slide at front and at rear side of said xy-block, whereas the y-rail block has a thread to interact with the threaded y-knob that drives the y-rails block relatively the xy-block with a brim, having further a threaded hole and the threaded x-knob placed into, sliding inside the distal slot of hinge;

8. The jig of claim 7 wherein the y-rails block has a set of guiding holes, perpendicular to a plane determined by the y-rails moves, to accept tools such as an aiming and a drilling guides, whereas a portion of the y-rails block, having said guiding holes, could be made detachable to comply with different intramedullary nails;

9. The jig according to 8 wherein detachable portion of the y-rails block has a bore between adjacent holes with flat coiled spring placed inside, whereas the outer diameter of the bore cut inside said holes letting the spring to protrude inside the guiding holes;

10. The jig according to 9 wherein the aiming guide comprised a rod with a central hole to accommodate a metallic spike or a drill and a disk, placed at one of ends of said rod, with radiopaque ring at an outer diameter whereas the rod and the disk made of radiolucent material and cuts made along said rod whereas the disk placed between the radiolucent device and the jig.
